(19) United States
(12) Patent Application Publication
(10) Pub. No.: US 2025/0285731 A1
(43) Pub. Date: Sep. 11, 2025

(54) DRUG DELIVERY DEVICE WITH HIDDEN MARKING

(71) Applicant: Bristol-Myers Squibb Company, Princeton, NJ (US)

(72) Inventor: Martin John McLoughlin, Hillsborough, NJ (US)

(21) Appl. No.: 18/858,178

(22) PCT Filed: Apr. 24, 2023
(86) PCT No.: PCT/US2023/019620

§ 371 Date: Oct. 18, 2024

Related U.S. Application Data
(60) Provisional application No. 63/333,809, filed on Apr. 22, 2022.

(51) Int. Cl. G16H 20/17; A61M 5/142; A61M 5/20; A61M 5/24; G06K 7/10

(52) U.S. Cl. CPC G16H 20/17; A61M 5/14248; A61M 5/20; A61M 5/2422; G06K 7/10861; A61M 2005/206; A61M 2005/208; A61M 2202/0007; A61M 2205/584; A61M 2205/6072; A61M 2205/6081

(57) ABSTRACT

The subject invention is for use with various drug delivery devices, including medical injectors, medical pill bottles, and medical pill blister packs. The subject invention utilizes one or more optically readable codes which are initially concealed and, later revealed during or after use. The encoded data of the optically readable codes may be obtained to provide information for various purposes, including, but not limited to, verification of proper drug and dose for a given patient, dosing duration, and successful completion of dosing.

### CROSS-REFERENCE TO RELATED APPLICATIONS

This application is a National Stage Application under 35 U.S.C. § 371 of PCT Application No. PCT/US2023/019620, filed Apr. 24, 2023, which claims the priority benefit of U.S. Provisional Application No. 63/333,809, filed Apr. 22, 2022; the contents of which are herein incorporated by reference in their entireties.

### FIELD OF THE INVENTION

The field of the present invention is the assurance of compliance with an administration regimen in the self-administration of drugs.

### BACKGROUND TO THE INVENTION

Recent advances in biotechnology and medicine have led to a large expansion in the number of biotechnology derived therapies such as monoclonal antibodies for the treatment of a broad range of conditions.

For a variety of reasons, such drugs must be administered parenterally because as therapeutic proteins they would be destroyed by the digestive system if administered orally, rendering them ineffective. Therefore, such biologic drugs are typically delivered via routes of administration that bypass the digestive system, most typically via the intra-venous and sub-cutaneous routes.

Many of the diseases treated by such drugs are chronic in nature and require treatment over periods of months or years. In many cases patients will remain on such treatments for life. Consequently, to minimize patient burden there is a strong preference to formulate such drugs as liquids for sub-cutaneous administration. In contrast to intra-venous formulations which must be administered in specialist clinics by trained healthcare professionals with all of the attendant logistic and time intensive considerations, sub-cutaneous formulations can be provided in a form suitable for self-administration by patients in the home. To support this trend, in tandem with the development of the sub-cutaneous drugs themselves, there has been in recent years a large expansion in the development of injection device technologies designed to further simplify the injection process, with particular emphasis on self-injection. Such devices include pre-filled syringes, pen injectors, auto-injectors and on-body injectors. These technologies are typically pre-filled with the sub-cutaneous drug formulation by design and therefore eliminate the need for patients to transfer the liquid drug from vials which is otherwise time consuming, wasteful and a potential source of medication errors. The large majority of such devices are single use disposable in nature though re-usable devices do exist. These technologies are not only beneficial to the patient in terms of convenience and reduced burden of treatment, they can also reduce treatment costs by moving treatment to an outpatient setting.

Despite the aforementioned benefits to the patient and healthcare providers of self-administration in the home setting there are nevertheless some associated drawbacks and challenges. Leading amongst these are the risks of medication errors, due to either user error or device malfunction, and non-compliance with the treatment regimen. Non-compliance can be inadvertent or deliberate, for example patients may simply forget to take their drug, or may avoid taking it, for example, to avoid a negative side-effect. A further issue is that many patients suffer anxiety at first when performing an injection on themselves. Such anxiety may result from fear of needles, fear of making an error or simply the absence of the reassuring presence of a healthcare professional. Although many of these issues exist also with more conventional oral medications such as tablets and capsules, these issues may be exacerbated in the case of biologic drug injections by the more invasive nature of the route of administration, less frequent dosing schedules which, for example, can make it easier to forget when a dose is due, and the often high cost of the drug which can heighten anxiety over the cost of making an error.

For these reasons, there has in recent years been significant growth in interest in providing technological solutions to address these problems. Such developments have in large measure been made possible by the remarkable recent progress in electronics, wireless communication, battery technology and portable computing which have driven miniaturization and cost reductions to the point where it is now feasible to integrate such technologies into injection devices. Using these technologies it is now possible for an injection device to have a secondary function as a remote networked computer terminal which is guaranteed to be present at the point-of-care. Typical systems incorporate minimal low cost electronics into the injection device itself, or an accessory to the injection device, and rely on the data display and wireless communications capabilities of the patient's smartphone to provide a user interface and to perform the data transfers. For example, a mobile app may be provided which interfaces with the injection device by a local or personal area networking technology such as near field communication (NFC), wifi (IEEE 802.x) or Bluetooth to create a wireless ‘tether’. By means of such a tether the device and the smartphone application can synchronize in order to provide the patient with information such as device status, injection progress or context dependent instructions via the smartphone display. The wide-area remote networking capabilities of the smartphone via cellular or wifi technologies can then perform remote network access for data transfer to remote services such as a physician portal, electronic health record or similar. Such data may include a record of the injection including patient identity, drug batch number, device serial number, time and date, geolocation (obtained from the smartphone's GPS location service) and error information. Error information may include internal error status flags, for example, from sensors integrated into the device such as an injection completion status flag. In this way a complete local area/wide area device communication system can be designed. Implementing functionality on the user's smartphone is beneficial in minimizing the amount of bespoke electronic hardware that is required to realize the system and may enable the minimal device electronics to be single use disposable also.

However, despite the great progress in miniaturization and cost reduction in the electronic technologies, there are nonetheless cost, complexity and sustainability concerns associated with use of electronics, especially disposable electronics. In response some manufacturers of injection devices have opted for re-usable electronic accessories for single-use disposable prefilled injection devices. Typically such accessories incorporate minimal electronics and mechanically couple to the pre-filled injection device. By means of sensors of types known to the person of ordinary skill in the art such accessories can determine the status of the pre-filled injection device and communicate via wireless communications protocols in the aforementioned manner. Examples of such devices may be found in U.S. Published Patent Appl. No. 2014/0194826A1, U.S. Published Patent Appl. No. 2019/0217022A1, and, U.S. Pat. No. 10,792,441B2.

Although such re-usable electronic accessories circumvent the aforementioned cost and sustainability issues, they do have drawbacks of their own which include the addition of more user steps, such as physical coupling to the injection device, and additional complexity in the product supply chain as there now may be two parallel supply chains for the pre-filled injection device and the electronic accessory. The supply chain for pre-filled injection devices typically requires cold-chain logistics to maintain the drug at the appropriate storage temperature, typically in the range 2-8° C. The electronic accessory on the other hand does not require cold chain logistics and since it is re-usable, there is no one-to-one correspondence with the prefilled injection device which would support co-packaging of injection device and accessory. Furthermore, for reasons of sustainability, a reverse logistics supply chain to recycle and or reclaim post market electronic waste may be desired or mandated by regulations in some territories. These considerations create complexity and costs that must be borne by the actors in the healthcare system.

Despite the potential benefits of improved medication compliance, previously the availability of smartphones restricted the creation and adoption of such systems however this is no longer an issue in developed countries due to the ubiquity of smartphone technology and this trend is likely to continue. Nevertheless some of the cost and sustainability issues have stalled adoption of these technologies.

Lower cost alternatives to the type of system described have also been proposed based on machine readable labels. Such systems rely on the capability of smartphones to read optical or radiofrequency labels such as bar codes, quick response (QR) codes, NFC or RFID tags. Typically an optical or radiofrequency label is applied to the injection device or the outer packaging at the point of manufacture. The label encodes product identifier information such as identity, strength, batch number, expiry date and possibly a unique serialization code. When ready for use by the patient, a dedicated smartphone application, each instance of which is uniquely associated to an individual patient, is used to read the information stored on the label by means of the smartphone's built-in camera for optical labels or the radio transceiver for radiofrequency labels. The combination of label data from the injection device and the unique identifier of the patient's smartphone application, along with the system date and time, and possibly geolocation, can be used to generate a packet of data that provides a timestamp for the date, time and location that a specific injection device was scanned by a specific user. By means of the smartphone's wide area networking capability this packet of data can be transmitted to a remote database where it can be used for purposes such as compliance monitoring, feedback, safety monitoring and so on. For example, a physician could access the database by means of a web application to check on the patients under their care. Other features of the smartphone application may include the ability for the patient to record symptoms or upload comments and feedback for the physician.

A limitation of this simple system is that the reading of the label and the actual use of the device are asynchronous meaning that there is no guarantee that the label read and use occur at the same time. In fact, the label read alone does not confirm that the device is used at all, only that the label has been read. This clearly limits the applicability of this technology for compliance monitoring purposes.

U.S. Pat. No. 10,980,948 provides a medical injector with a display which is blank in an initial, pre-use state, and onto which, a bar code is generated and displayed post-use. The bar code is scannable to provide indication that the injection was completed.

### SUMMARY OF THE INVENTION

The present invention improves on known simple monitoring technology by use of at least first and second optically readable codes on a drug delivery device, which may be in the form of a medical injector (e.g., a pen injector, a syringe, a body-wearable injector), a medical pill bottle, a medical inhaler, and, a medical pill blister pack. The first optically readable code may be applied as known in the art to an external portion of a drug delivery device. The second optically readable code is concealed before use or accessing of a dose of medicine and revealed during or following use. This enables discrimination between a ‘before use’ state and an ‘after use’ state. In one approach, the invention exploits mechanical features of a medical injection device by placing the second optically readable code on a component which forms part of the injection mechanism and is displaced during the injection from a concealed state to a revealed state. Most injection devices of the single-use pre-filled type have a viewing port or window to enable the visual inspection of the drug solution prior to injection. In many devices known to the person of ordinary skill in the art the window is progressively obscured by a travelling component of the injection mechanism as the injection proceeds. In many devices this function is used to provide an injection progress and/or status indicator. For example, the obscuration of the window by an opaque component can be used to provide a simple visual check that the device has already been used to avoid the error of mistaking a used device for an unused one.

In a further approach, the subject invention exploits the features of drug containers, in pill-bottle or blister-pack form, where a portion of the drug container needs to be adjusted or removed to expose the second optically readable code. Although user intervention is required for the revealing of the second code, advantageously, the user's normal interaction with the drug container will cause the revealing.

These and other features of the invention will be better understood through a study of the following description and accompanying drawings.

### BRIEF DESCRIPTION OF THE DRAWINGS

FIGS. 1-9 show a medical injector as a pen injector in accordance with the subject invention;

FIGS. 10-11 show a medical injector as a syringe in accordance with the subject invention;

FIGS. 12-29 show a medical injector as a body-wearable injector in accordance with the subject invention;

FIGS. 30-33 show a medical pill bottle in accordance with the subject invention;

FIGS. 34-37 show a medical pill blister pack in accordance with the subject invention; and,

FIG. 38 shows a system useable with the subject invention.

### DETAILED DESCRIPTION OF THE INVENTION

The subject invention is for use with various drug delivery devices, including medical injectors, medical pill bottles, and medical pill blister packs. The subject invention utilizes one or more optically readable codes which are initially concealed and, later revealed during or after use. The encoded data of the optically readable codes may be obtained to provide information for various purposes, including, but not limited to, verification of proper drug and dose for a given patient, dosing duration, and successful completion of dosing.

In one embodiment, the first optically readable marking is applied to the body of the drug delivery device, for example as part of the product label. The data encoded on the first marking may include product identity (e.g., drug or drugs), strength of the drug, manufacturing batch number, time and/or date of manufacture (date of manufacturing the drug; time and/or date of assembling the drug delivery device), expiry date of the drug and a unique serialization number.

The second optically readable marking is applied to the drug delivery device so that prior to use said second marking is concealed from view. Use of an optically readable code ensures the second code can only be read when there is a line of sight to the second code. The second marking is only visible following a drug administration, e.g., after an injection where the travelling part (e.g., plunger) has completed its travel to expose said second marking. Optionally, the data encoded on the second marking may include a unique number which is algorithmically related to a unique number encoded in the first marking. The second marking may be used alone, without any further optically readable markings.

As used herein, “optically readable code” (also referred to as “optically readable marking” or “marking”), and derivatives thereof, refer to data in a structured format that can be optically read by a computer and processed without human intervention, such as in the form of bar codes, quick response (QR) codes, data matrices, and the like. In addition, this definition refers to printed machine readable numeric or alphanumeric characters, which may be especially suitable where the area available for encoding a code, such as with QR codes, may be too small to be reliably marked and read. In such cases, a unique printed number or string may be directly marked on the drug delivery device in machine-readable typeface which is also readable by a user (i.e., the printed number or string include the actual numbers). Such typefaces are known to the person of ordinary skill in the art and are in widespread use in financial documents such as checks and credit cards. Examples are OCR-A, OCR-B, MICR E13B. An advantage of providing the first and/or second marking in a printed numeric or alphanumeric form is that it is both human and machine readable. This enables the possibility of manual entry of the code into the application software as a back-up option for situations where the machine vision function of the user's device has failed to read the code, for example, as a consequence of poor illumination, limited camera resolution, distortion due to refractive surfaces or loss of integrity of part of the marking rendering machine vision impossible, and the like.

Further, as used herein, “drug” refers to one or more therapeutic agents, including combinations or mixtures with other components such as diluents, carriers, and the like.

The first and second optically readable codes may be applied using any technique, including, but not limited to: being directly printed (e.g. using inkjet printing, laser printing) on a portion of the drug delivery device (preferably in sufficiently contrasting color to allow optically reading thereof) being formed or etched on a portion of the drug delivery device (with possible subsequent coloring or painting to provide sufficient contrast for optical reading); and/or, being printed on a label which is applied to the drug delivery device, such as by adherence with the label having pressure sensitive adhesive.

In a further aspect of the invention, there is provided dedicated application software downloadable for use on a camera-equipped, Internet-enabled device, which may be referred to herein as a “user's device,” such as a smartphone, tablet, laptop, and the like. The application software is programmed to use the user's device's camera to capture images of the drug delivery device. Optionally, the application software, may be configured to generate a user interface on the user's device which may provide on-screen guides to guide the user to capture images with an appropriate field of view. In embodiments, the application software may include machine vision algorithms (e.g., algorithms for one or more of pattern matching, template matching, feature matching) for the recognition of certain features of the drug delivery device, for example to ensure that the device status is correct for the specific step and to provide protection from malicious attempts to fake the captured image. For example, the application software may be designed to measure relative distances between the second marking and the first marking or some other appropriate datum point on the drug delivery device in order to provide additional confirmation of correct use and an elementary form of error detection. With the drug delivery device being a medical injector, this may be used to detect when the travelling part has not completed its full stroke which may be indicative of a device malfunction, e.g., caused by a mechanism failure or a needle occlusion. Features on the drug delivery device or marking(s) may be provided to provide a distance calibration to compensate for the variable distance of the user's device from the drug delivery device. Should protection from malicious actors attempting to ‘spoof’ the application software into registering spurious use confirmations, for example by arranging fake markings to pass off as real markings in an attempt to discover the secret algorithmic relationship, the measurement of relative distance could be used to confirm correct registry of the markings as an elementary form of ‘anti-spoofing’ measure. In alternative embodiments, positional markings may be provided directly to the drug delivery device, e.g., on the travelling component.

Each instance of the application software may be uniquely registered to individual users with a numeric encoding or user ‘certificate’ or ‘key’ which is registered with a service provider so that collected drug-administration data may be resolved at the individual patient level if desired. This may provide a user identification useable for association with collected data which is transmitted for review and analysis. The user identification may be assigned to a user (through various channels, e.g., by mail, email, hand deliver), generated by the user's device upon downloading the application software, and/or by the application software, once downloaded. The user identification may be related to an identification associated with the user's device (e.g. using a unique International Mobile Equipment Identity (IMEI) associated with the user's device). Means for establishing trust by means of digital certificates and public key encryption are well known to the person of ordinary skill in the art and may be implemented in the application software to confirm identity, sign data transfers and so on.

As an example, a user removes the drug delivery device in the form of a medical injector from its packaging and launches and signs in to the application software downloaded on the user's device. The user is prompted to capture an image of the unused injection device from which the optically readable data stored in the optical encoding of the first optically readable marking can be extracted for processing by machine vision algorithms. The image and the extracted data may be stored with a timestamp provided by the operating system of the user's device or a remote time server, recording the time and date of image capture. Prior to use of the injection device, the application software may be also programmed to confirm the absence of the second marking by means of machine vision algorithms to confirm that the device is unused. The patient is then prompted to perform their injection as usual. Following the injection, the application software may be used a second time to capture an image of the now used injection device. From this second image, the data from both the first and the second markings are extracted and the image and data are stored with a new timestamp. The time interval between the first and second images identifies the time interval in which the injection was performed.

Any data captured by the application software may be transmitted (e.g., wirelessly) to a remote server for storage and processing. The data, subject to privacy laws and regulations, may be made accessible to one or more of: the user's physician, the manufacturer of the administered drug, the manufacturer or assembler of the drug delivery device, an insurer, and a third-party compliance organization.

Applicant has realized that the integrity and security of such a system, and therefore its ability to function as a compliance monitor, is improved if the unique numbers stored in the first and second markings are related and together provide a unique ‘fingerprint’ of the individual device in both the unused and used states to provide positive verification that the specific device has been successfully used. In the case where the unique numbers on the first and second markings are unrelated, the system is unable to detect errors or misuse by users and could be fooled by a malicious actor replacing labels from other devices or forgery of labels, for example. By relating the unique numbers, such attempts to do so would be detected and reported as errors or misuse. For security and integrity of the system, the relationship between the numbers should be secret and not be so simple that they are discoverable by users or other actors in the use of the device. For example, if the numbers on the first and second markings were the same, the security of the system could be easily compromised because the concealed second marking would be known prior to use. The relation between the unique numbers with adequate security could be achieved by generating random numbers and maintaining a look-up table or database of the pairs of said numbers used for the respective markings on each device at the point of manufacture.

Therefore, in a further aspect of the invention, an algorithmic relationship may exist between the unique numbers stored on the first and second markings such that the number stored on the second marking is the result of said function operating on the number stored on the first marking. The exact algorithmic relationship may be secretly encoded in, or accessible by, the application software and known only to the system manufacturer. Once these numbers are captured from the first and second markings respectively, using one or more algorithmic operations, the application software may compute the result of the function operating on the number stored in the first marking to generate an intermediate code. Once the intermediate code is computed, the result is compared with the number encoded on the second marking. If the intermediate code matches that encoded in the second marking, then it is confirmed that the first and second markings are valid and the application software records successful injection within the time interval of the first and second image captures. A further benefit of the confirmation of valid codes is that it provides an elementary form of anti-counterfeit protection.

In order to implement the algorithmic relationship in the first and second markings, the algorithmic operation must be performed at the point of manufacture on the unique number encoded in the first marking and the result encoded in the second marking. This requires the ability for variable marking at the point of assembly of the drug delivery device. This may be achieved by high speed marking technologies such as laser marking that are known to the person of ordinary skill in the art.

As will be appreciated by those skilled in the art, the application software may be configured to generate the intermediate code based on the second marking (after revealing) for verification against the number encoded in the first marking.

The specific algorithmic relationship used to relate the first and second markings may be of varying complexity. In order for this scheme to produce a unique fingerprint for each device, the function should be single-valued such that there is a single unique numerical result from the operation of the function on the inputs. More complex functions will provide greater protection from attempts to discover the secret function however there are constraints on the selection of the function related to the data storage density of the optical encoding and the size of marking which places an upper limit on the magnitude of the numbers that can be reliably stored. Optical encodings such as QR codes can store data in different formats, such as character, numerical, binary, and can utilize varying degrees of error correction by means of redundancy which reduces the amount of data that can be stored. The selection of an optimal storage format requires trade-offs between the degree of error protection, the available area for the marking, camera resolution and amount of data to be stored. It is preferred that the inputs and result of the function are integers to minimize the size of the optical encoding required to store them.

For purposes of illustration, a very simple algorithmic relationship may be simply the subtraction of the unique number of the first marking from a constant number which is secretly encoded in the application software. If the number of the first marking is an integer A and the constant is an integer C, then C−A=B, where B is the integer result (the intermediate code) which matches the number encoded in the second marking. C may be arranged to be larger than A so that the result B is a positive integer so that sign can be disregarded. The magnitude of the respective numbers will be determined by the encoding constraints of the optical markings used. If the unique number encoded in the first marking is also to be used as a unique serialization of production units then the magnitude must be chosen to be sufficient for unique numbering of all anticipated production. Since there will typically be less area for the second marking (due to concealment constraints) than on the device body for the first marking, it is preferable to arrange the numbers so that the result to be encoded in the second marking requires less optical storage than the first.

For the purposes of illustration with specific example numbers, suppose that A is the randomly chosen base 10 integer 21432364 and C is 25432449 (also base 10 and randomly chosen). Then the result C−A=B=4000085 is the number to be encoded in the second marking. This calculation is performed at the point of assembly by the assembly equipment and the result encoded into the second marking which is applied to the travelling component. At the point of use, the inverse function is performed by the application software, which is the addition B+A. If the result of the addition equals C then the codes are valid and confirmed. In the example, 21432364+4000085=25432449 and the result is confirmed.

More generally, the algorithmic relationship f between the first marking A and constant C is f(A)=C−A. The inverse function g is g(A,B)=B+A. Note that the encoded numbers are not limited to base 10 and other bases such as binary, octal or hexadecimal may be used and may allow for more efficient optical storage. It is to be understood that the foregoing is provided for purposes of illustration only and is one example of an infinite number of possible algorithmic relationships which can be utilized within the constraints described above. Nonetheless, despite its simplicity, assuming the constant C is selected randomly and kept secret then the degree of validity confirmation may be good enough for most normal purposes even with such a simple function. However, for counterfeit protection, the algorithmic relationship in this case is easily discoverable if a counterfeiter can obtain multiple samples of used devices, therefore greater complexity of algorithmic relationship is preferred for stronger anti-counterfeiting measures.

In embodiments, the secret function may be an encryption cipher which converts some or all of the data stored on the first marking into encrypted form which is written to the second marking. In the terminology of cryptography, the data from the first marking intended for encryption would constitute the ‘plaintext’ and the encrypted data intended for writing to the second marking would constitute the ‘ciphertext,’ or vice versa. In broad terms, encryption mechanisms are divided into ‘symmetric’ and ‘asymmetric’ methods. Symmetric methods require a single password, passphrase or ‘key’ whereas asymmetric methods implement so called public key encryption and require two keys, one public and one private. Standard algorithms for both types of encryption are known to the person of ordinary skill in the art and are in widespread use. Generally, asymmetric encryption provides stronger encryption and also the possibility of using electronic signatures to sign data; however, they are more complex, requiring the management of public and private keys. Among the advantages of using standard encryption ciphers as the secret function is that they typically use a random seed in the generation of the ciphertext which makes the cipher more secure and in the context of the present invention makes discovery of the secret function more difficult. However, among the drawbacks of such algorithms are that ciphertexts may be substantially larger than the plaintexts from which they are generated. Furthermore, because of the random seeding of the encryption algorithm, the length of the ciphertext may be unpredictable. Because of optical encoding constraints arising from limitations on the available area for applying the second marking to the travelling component and limits on marking technology precision and camera resolution, the unpredictable size of the ciphertext may prevent encoding the ciphertext in the second marking. Simpler encipherment algorithms are known such as simple substitution algorithms in which the plaintext and ciphertext are guaranteed to have the same length, however, these may not offer more protection to secret function discovery than the aforementioned trivial example. Encryption of the optically encoded data is not strictly required for the present invention. Instead, a unique pairing of codes to provide a fingerprint of the used and unused states of an individual device is required. This requires only maintaining the algorithmic relationship between the two markings secret. Since the algorithmic relationship is not encoded directly in either marking, encryption is not strictly necessary and so the benefits of standard encryption algorithms may be outweighed by these drawbacks when used as the secret function.

Preferably, however, the algorithmic relationship should be difficult to discover to avoid compromise of the system integrity. To address this, in further embodiments elements of other data fields stored on the first marking such as the date and/or time of manufacture and manufacturing batch number may be used to create stronger secrecy in the algorithmic relationship because the use of variable inputs instead of a constant as described in the prior example introduces an element of randomness or pseudo-randomness which makes the secret function more difficult to discover. An example of how this may be achieved is by the use of cryptographic hashing algorithms. An ideal hashing algorithm is designed to produce a unique output, called a ‘digest’ or ‘hash’, for a given input. Hence hashing algorithms may be regarded as single-valued functions in the strict mathematical sense. This feature makes such algorithms suitable for creating a unique ‘fingerprint’ of the input data which is useful for checking integrity of data because any modification to the input, either inadvertent, for example due to transmission errors, or deliberate such as tampering by malicious actors, results in a different fingerprint. A further feature of hashing algorithms is that they produce a fixed length output string irrespective of the length of the input. This feature is helpful for optical encoding as the number of bits of data to encode, and therefore the size of the optical code, is always fixed and known in advance. A final feature of a hash is that it is essentially impossible to reproduce the data from the hash itself. In effect this means there is no inverse hash function that can be used to retrieve the data and assist in discovering the secret function as in the simple subtraction function example above. Hashing algorithms are widely used in computing and cryptography and are known to the person of ordinary skill in the art. Some well-known hashing algorithms are the MD4, MD5, SHA1 and SHA256 algorithms.

Despite the reliable reproduction of the hash from a given input, the hash itself is an obscure alphanumeric string which appears random and meaningless. This feature makes hashes suitable for obscuring input data to prevent discovery of the secret encoding scheme.

To use hashing algorithms in the present invention, it is not sufficient to simply generate a hash from the data stored on the first marking and write this to the second marking because the data on the first marking are available prior to use and in principle the hash, and therefore the marking, for the second label could be correctly and reliably generated prior to use of the device therefore enabling a ‘false use’ to be recorded by the application software for an unused device. Therefore, there is still a need to perform a secret mathematical functional or other conversion operation on some or all of the data of the first marking. However, once the result is computed, said result is passed through a hashing algorithm with some or all of the other data on the first marking to generate a hash which is written to the second marking. This produces a reliable but undiscoverable algorithmic encoding between the first and second marking. Assuming that some of the data encoded in the first marking is variable and semi-random, such as the time of manufacture, then the secrecy and security of the hash is enhanced further. Furthermore, because the hash is always of fixed length, the issue of ciphertext size vs plaintext size described above when encryption algorithms are used as the secret function is negated. Therefore, in one embodiment the second marking may encode the hash of the ciphertext of the first marking. The key for the encryption algorithm would be maintained as a secret in the smartphone application.

In still further embodiments, Applicant has realized that it may be advantageous for the application software to generate hashes of the images captured of the unused and used devices, for example, to address the possibility of ‘double-captures’ in which the user or a malicious actor captures multiple images of the same device in a used or unused state and attempts to re-use them. The hash generated from an image file can be considered a unique fingerprint of the image that uniquely identifies an image. This relies on the hashing algorithm's sensitivity to the input data. For all practical purposes, it is an impossibility for two distinct image captures of the same scene, to be sufficiently identical that a hashing algorithm would produce the same hash as a result. For example, the slightest difference in lighting causing differences in the color and intensity of a small number of pixels will be sufficient to change the hash result resulting in different fingerprints. If the image files from which the hashes are generated include metadata such as EXIF data which records time and date of the image capture for example, then the files are guaranteed to produce different hashes. The images and computed hashes can then be used in the smartphone application and in the extended system to provide a high degree of verification of the veracity of the image captures and their time and date of capture. If geolocation is enabled in the camera application and is recorded in the image metadata then location can also be verified.

It is noted that a first number, encoded in the first optically readable code, and a second number, encoded in the second optically readable code, may be algorithmically related. As discussed above, one or more algorithms based on mathematical operations and/or encryption techniques may be used to relate the first and second numbers. The mathematical operations may be one or more basic operation involving a predetermined value (such as constant C discussed above) including, subtraction of the predetermined value, addition of the predetermined value, multiplication by the predetermined value, and division by the predetermined value. As discussed above, the algorithm(s) may be programmed into the application software along with any necessary predetermined values.

In addition, the application software may be configured to decode a data bit or data string from one or both of the first and second markings to obtain a key for determining the algorithmic processing to be conducted. In this arrangement, the application software may have a look-up table associated therewith in which a plurality of algorithmic operations is associated with various keys. During manufacturing, a key is selected based on the algorithm(s) used in determining the second number with the relevant key being encoded in the first and/or second marking. Upon decoding the first and second markings, the key is obtained and used to determine the relevant algorithmic operation(s). This allows for a level of randomness in application of algorithms. As a further alternative, the first or second marking may include a link (e.g., a IPV6 Internet address or the like) to a remote database having stored therein a plurality of algorithmic operations associated with various keys. The application software may be configured to call the remote database with the decoded key to identify the relevant algorithmic operation(s).

When aggregated the data stored on the first and second markings, the result of the algorithmic confirmation, the time interval between the image captures and the user ‘key’ provide confirmation that the device was successfully used within the time interval recorded, by a specific user without any electronics beyond the user's device itself. The data can subsequently be transferred to a remote database managed by a service provider and is amenable to further analytics. Analytics may include adherence to a dosing schedule or device malfunctions (such as incomplete injections). As one example of such analytics, long time intervals between the first and second image captures may be indicative of a user having difficulty who is in need of extra training. The healthcare provider may be alerted to this trend to enable appropriate intervention.

In further embodiments of the invention, still further concealed markings may be applied to other parts of the drug delivery device which are concealed in the unused state and revealed in the used state. For example, some injection devices have a needle shield that is deployed at the end of the injection and locks in place to prevent needle-stick injury from used devices. In such devices, a third marking may be concealed on the hidden part of the shield component to be revealed when deployed. Now, instead of a pair of encodings on first and second markings, there is a triplet of encodings that may be algorithmically related. Alternatively, this can be seen as a one-to-many relationship between the first marking and the hidden (second and third) markings. In principle, this can be extended to any number of hidden markings resulting in an n-tuple of encodings, however in practice there will be limitations on how many such encodings can be used. The mathematical definition of a function requires that it be single valued, a feature that was exploited in the first described embodiment comprising a first and second marking in order to ensure that a unique result was obtained. Though not strictly functions in the mathematical sense, multi-valued functions are known however there are likely to be too many constraints on their use in the implementation of one to many relations between encodings. In many cases where multiple hidden markings may be used they will be revealed as part of a defined sequence of actions. For example, in an injection device with deployable needle shield, the first marking is visible when the device is removed from its packaging, the second marking is revealed when the injection is completed, and the third marking is revealed when the needle shield is deployed. This sequence of actions can be encoded by a sequence of applications of the same hidden function in which the same function is applied to the results of the previous application of the function in sequence. Such ‘chaining’ together of functions ensures that a strict sequential functional relationship is maintained between the encoding on each marking in the chain. As in the original example of two markings, the algorthmic relationship should not be discoverable. Indeed, in the case of more than two markings, this requirement is stronger because now multiple results of the application of the function are present on a single device.

To provide a trivial example of how this chaining of functions might work reference is made to the previous example of a simple subtraction function in which the function f(A)=C−A=B where C is a secret constant. In the case of three markings, the encoding of the third marking is obtained as f(B)=C−B=D. More generally, if the number encoded in the ith marking is Xi then f(Xi)=C−Xi=Xi+n. As before, this example though illustrative is likely to be too simple to fulfill the requirement of being undiscoverable. To provide an undiscoverable chain of encodings, hash functions may be chained together to generate a ‘hash chain’ in which, starting with seed data obtained from data encoded in the first marking and the operation of a secret function thereon, a hashing algorithm is applied and the result written to the second marking. This procedure is repeated as many times as there are markings required such that the result of the first application of the secret function and hash algorithm is written to the second marking and becomes the input for the next application of the secret function and hashing algorithm and so on until all markings have been encoded.

Symbolically, if the hash function is h, the secret function is s and the seed data S, then the first hash encoded in the second marking is h(s(S)), the second hash encoded in the third marking is h(s(h(s(S))) which can more conveniently be written h\hat{ }2(s\hat{ }2(S)). In the general case, the nth hash encoded into the n+1th marking is h\hat{ }n(s\hat{ }n((S)). Because hash functions do not have an inverse, the verification of the veracity of the nth hash read from the nth marking is to compute h(s(Sn−1)) where Sn−1 is the hash stored in the n−1 th marking and to compare with the hash read from the nth marking.

To apply the markings to the travelling components in such a scenario would require that the components be marked in accordance with the above encoding scheme such that the chain of hashes would be computed in-line during the assembly process and applied to the respective components in an extension of the two-marking scheme.

With the drug delivery device being a medical injector, there may be reliance on an automated movement of the device to reveal the second marking, particularly as part of the injection, such as with movement of the travelling part. In different forms of the drug delivery device, the revealing may be caused by manual action of a user, e.g., in opening a pill bottle or opening a sealed blister of a blister pack. With this type of arrangement, the second marking is concealed and normal manual interaction of the user is utilized to reveal the second marking, e.g., with manual opening of the pill bottle and manual opening of a blister of the blister pack. It is further possible to provide the second marking with a peelable cover which is removable by a user when peeled to reveal the second marking. With this arrangement, the second marking may be located on external portions of the drug delivery device, without need for physical concealment by a portion of the drug delivery device. It is additionally noted that the details of this paragraph apply to all concealed, to-be-revealed markings (such as the above-discussed third marking). Modes of concealment may be mixed with two or more concealed markings, e.g., with automated revealing of a second marking with movement on a medical injector and manual peeling of a cover on the injector body to reveal a third marking. The revealing of the second marking alone may be utilized, without use of the first marking. The revealing of the second marking provides an indication of dose completion and may provide useful information without relationship with the first marking or any other optically readable codes.

In one approach, the process flow for manufacture in these scenarios may involve: the optical encoding of data to generate the first marking during the drug delivery device assembly process and applying that marking to an outer surface of the drug delivery device; the application of one or more algorithmic operations to generate data for the second marking; encoding and writing of said second marking; and, applying the second marking to the drug delivery device. It is noted that the first marking, applied to an outer portion of the drug delivery device, may be applied at various stages in the manufacturing/assembling process, including with the drug delivery device fully assembled. The second marking, as being concealed, may require application to a component of the drug delivery device before or during assembly, with necessary accessibility being available. For example, with the drug delivery device being a medical injector, the second marking may be applied to the travelling component (e.g., plunger), before or during the assembly, as the travelling component may be housed and be inaccessible post-assembly.

In some circumstances, it may beneficial to mark components of the drug delivery device with a unique number or encoding for the second marking, before final assembly, when in their bulk form, for example by laser marking as a terminal step in their fabrication which will typically be by injection molding. In the final assembly process, the number or encoding may be read from each individual component as it enters the assembly process. The secret function will then operate on this as input to generate the result (by application of the relevant algorithmic operator(s)) which will be written to the first marking for application on the outer body of the device. This approach has the advantages of avoiding the need to mark components with unique encoding for the second marking during the assembly process and that the result is written to the first marking which may have a larger data storage capacity. This approach, however, may place some constraints on the secret functions that can be used because the direction of encoding is now from the second (hidden) marking, to the first marking. For example, using variable data such as time and date in the computation of the result for encoding the first marking may not be possible due to limited optical data storage available in the second marking.

### Medical Injector

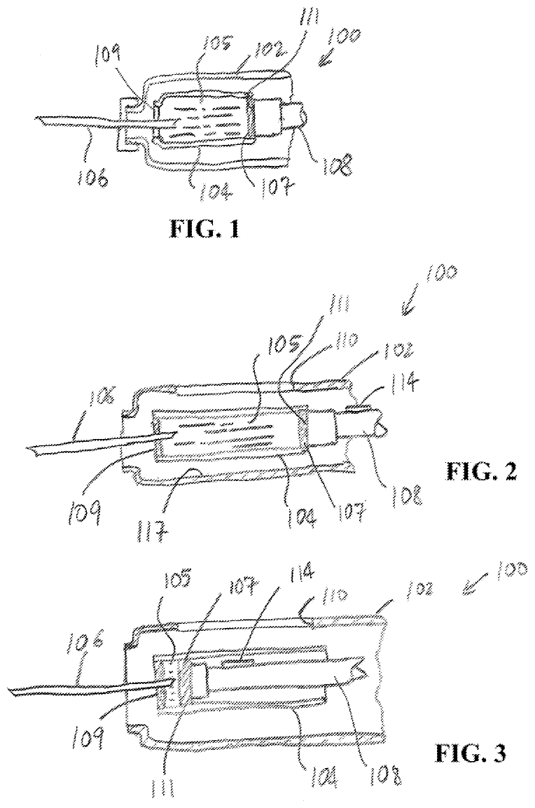

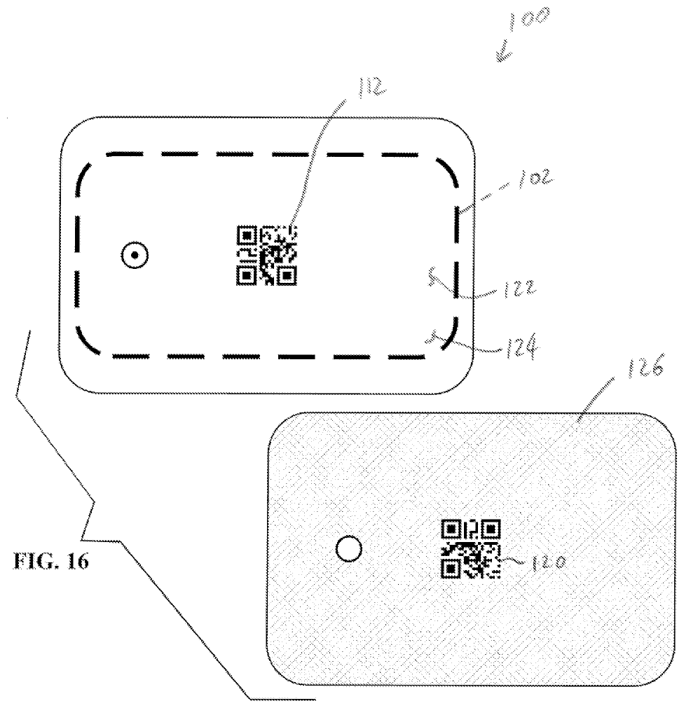

With reference to FIGS. 1-29, the drug delivery device of the subject invention is shown in the form of a medical injector 100.

The medical injector 100 may be of any known type. The subject invention is particularly well-suited to work with pen injectors. FIGS. 1-9 representatively show the medical injector 100 as a pen injector. The medical injector 100 may generally include a body 102, a drug reservoir 104 contained within the body 102, a cannula 106, and a movable plunger 108 for urging drug from the drug reservoir 104 through the cannula 106. The drug reservoir 104 is configured to accommodate a supply of drug 105. The drug reservoir 104 may be a stoppered ampoule as is known in the art. The plunger 108 may be configured to press against and displace a stopper 107 in the ampoule to urge drug therefrom, as shown in FIGS. 2-3. The cannula 106 may be pre-mounted to the medical injector 1004 or provided with mounting features, such as threads or a luer, configured to cooperatively mount to corresponding features on the body 102. The cannula 106 may access the contents of the drug reservoir 104 using any known configuration, such as, breaching a provided septum 109 on the drug reservoir 104 with mounting of the cannula 106 to the body 102.

The medical injector 100 may be provided as a single-use injector configured for a fixed dose, where a distal end 111 of the plunger 108 has a pre-defined length of travel. Alternatively, the medical injector 100 may be provided as a variable dose device, utilizing any known dose setting mechanism. With a dose being set, the distal end 111 of the plunger 108 is set to translate a distance set by the selected dose amount. The plunger 108 is shown schematically in FIGS. 1-3 as a representation. The plunger 108 may be of a fixed-length type, which is advanced by applying pressure to a proximal end thereof (the proximal end being away from the cannula) or by use of a threaded or gear drive (e.g., in the form of a leadscrew). Alternatively, the plunger 108 may be of an expanding-length type, such as a telescoping plunger which is caused to lengthen. The plunger 108 is configured to advance the distal end 111 in a distal direction towards the cannula 106.

The body 102 is provided with an open window 110 which allows visual access to the interior of the body 102. The open window 110 may be an open void, with no material therein, or provided with a clear or translucent lens. It is preferred that the body 102 be formed opaque so as to restrict visual access about the open window 110. A first marking 112 may be applied to an exterior portion of the body 102 outside of the open window 110. A second marking 114 may be located on the plunger 108 such that in a pre-use state, the second marking 114 is concealed by portions of the body 102, as shown schematically in FIG. 2. The plunger 108 and the open window 110 are configured such that the second marking 114 is revealed through the open window 110 with travel of the plunger 108 in urging the dose from the drug reservoir 104 through the cannula 106. The travel of the plunger 108 in delivering the dose shifts the second marking 114 from the concealed state to a revealed state visible through the open window 110, as shown in FIG. 3. FIGS. 1-3 are shown schematically, without all components of the medical injector 100, for simplification. As will be recognized by those skilled in the art, FIGS. 1-3 provide sufficient detail to convey the essence of the invention.

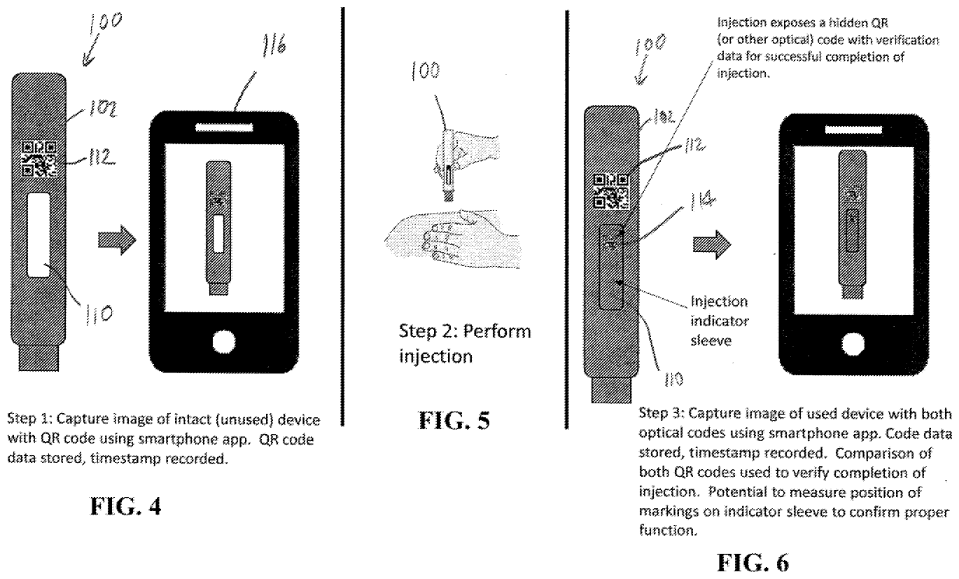

As shown in the FIGS. 4-6, the body 102 may be elongated to extend along a longitudinal axis. The first and second markings 112, 114, may be positioned along the longitudinal axis at spaced-apart locations. This allows for good alignment of the first and second markings 112, 114 in a common field of view of a camera of a user's device 116.

In use, as shown in FIG. 4, the user's device 116, having stored thereon the application software, may be used to capture an image of the medical injector 100 prior to use. The captured image of the medical injector 100 may be time stamped by the application software for reference. As discussed above, the application software may be configured to use machine vision algorithms, such as pattern matching, template matching, or feature matching, to confirm that the medical injector 100 is properly in a pre-use state. For example, the application software may be configured to recognize the open window 110 and expect to see a field of a particular color bounded by the open window 110. Coloration may be provided by the drug 105 to define the field of color, particularly with the drug reservoir 104 having a clear or translucent sidewall. In addition, or alternatively, with the drug 105 being clear or translucent, a portion 117 of the interior of the body 102, opposite the open window 110, may have coloration so as to be viewable as the field of color by the camera of the user's device 116. Full view of the expected color field provides indication of proper pre-use state with the plunger 108 being fully out of view. For example, as shown in FIG. 4, the field within the window 110 is fully shown as a single color (white). If the color field is partially or fully obscured, e.g., the field not being fully a single color, the application software may detect a possible error and provide indication to the user for manual confirmation.

Once the medical injector 100 is confirmed to be ready for use, as shown in FIG. 5, a medical injection may be performed. The cannula 106 may be initially covered by a needle shield 118 which is retractable with the pressing of the medical injector 100 against the site for injection. Alternatively, the cannula 106 may be exposed, un-shielded, prior to use, and inserted directly into a patient at the injection site. With the cannula 106 inserted at the injection site, any known arrangement for driving the plunger 108 for dosing may be utilized. As known in the art, the plunger 108 may be advanced by physical application of force by a user and/or by release of a motive force provided by a spring, compressed gas, and so forth.

As shown in FIG. 6, with completion of the injection, the plunger 108 resides in a post-use state with the second marking 114 being revealed through the open window 110 (as shown in FIG. 3). The user's device 116 may be used to capture a post-use image with the first and second markings 112, 114 being in a common field of view of the camera of the user's device 116. This captured image may be time stamped by the application software for reference. Similar to pre-use processing, the application software may utilize machine vision algorithms to confirm proper post-use state of the medical injector 100. For example, the plunger 108 may be provided in a color which contrasts the color of the original field of color. With image capture, the application software may view coloration within the open window 110 to compare the visible field of color with an expected field of color in determining whether the plunger 108 fully completed its travel.

The first and second markings 112, 114 may be decoded by the application software to read data captured therein. As discussed above, the first and second markings 112, 114 may include unique numbers which are algorithmically related, as discussed above. In this manner, the first and second markings 112, 114 may be collectively verified. Once verified, the application software may create a data packet, including the time stamps of the pre- and post-use captured images, along with indication of successful completion of injection, for transmission to a remote server for review and analysis.

It is noted, as discussed above, that the second marking 114 may be physically smaller than the first marking 112, particularly due to space constraints within the medical injector 100. As a result, the amount of encoded data may be less in the second marking 114 than in the first marking 112.

It is noted that glare or reflection may be present within the open window 110, particularly if a lens is provided, which may affect the ability to decode the second marking 114. The application software may be programmed to detect such anomalies and request or suggest repositioning of the medical injector 100 and/or the user's device 116 in seeking to establish a different sight line which may reduce or remove the glare or reflection.

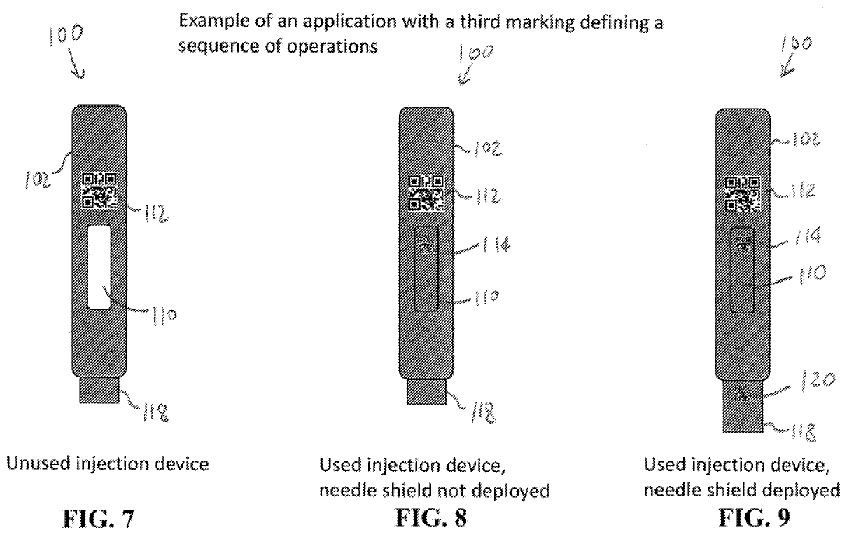

As shown in FIGS. 7-9 and as discussed above, additional markings beyond two may be used with the medical injector 100. The needle shield 118 may be provided with a third marking 120 which is concealed prior to use. Similar to the second marking 114, the third marking 120 may be obscured and concealed by a portion of the body 102. It is noted that the needle shield 118 may be fully retracted within the body 102 prior to use. With this arrangement, the needle shield 118 does not cover the cannula 106 prior to use. As discussed above, alternatively, the needle shield 118 may protrude from the body 102 to cover the cannula 106 prior to use. In either case, the third marking 120 is concealed within the body 102 prior to post-use shielding.

FIG. 7 shows the medical injector 100 in a pre-use state as discussed above, while FIG. 8 shows the medical injector 100 in a post-injection state as discussed above. FIG. 9 shows the needle shield 118 in a deployed state where the cannula 106 is shielded post-use. In the deployed state, the third marking 120 is revealed. It is preferred that the third marking 120 be located along the same longitudinal axis as the first and second markings 112, 114. In this manner, the user's device 116 may capture a post-use image of all three of the first, second and third markings 112, 114, 120 being in a common field of view of the camera of the user's device 116. Data may be decoded and utilized in the same manner as discussed above. A fourth, and additional, markings may be used, e.g., being located on an exterior of the body 102 with a peelable cover.

The needle shield 118 may be formed in any known manner, particularly to be released during or after the injection. The needle shield 118 may be spring-biased and advanced forward to cover the cannula 116 with release from a patient's skin at the injection site.

With typical pen injectors, the drug reservoir 104 is an ampoule. Drug is urged from the ampoule with insertion of the plunger 108 thereinto. The second marking 114 may be advanced into the interior of the ampoule with distal advancement of the plunger 108. The sidewall of the drug reservoir 104 may be formed clear or translucent to allow for visual sighting of the second marking 114 in the post-use state, within the drug reservoir 104.

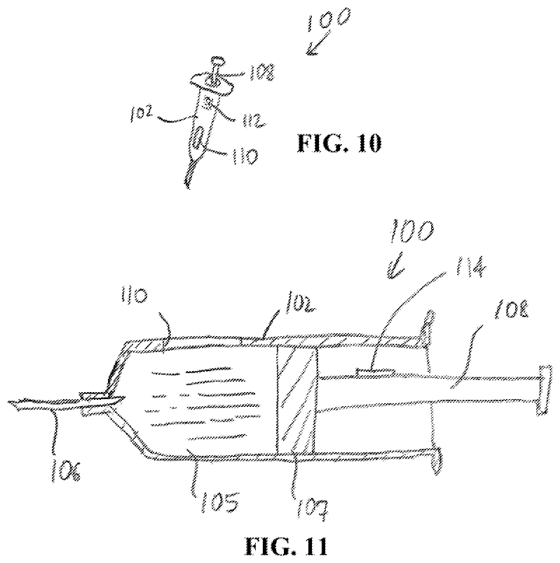

In an alternate embodiment, the medical injector 100 may be in the form of a syringe, as shown in FIGS. 10-11. The body 102 may be in the form of a barrel, with the drug 105 contained therein. The stopper 107 may be mounted directly to the plunger 108, with the stopper 107 being in sealing contact with the body 102. The stopper 107, in combination with the body 102, defines the drug reservoir 104. With distal advancement of the plunger 108, the stopper 107 urges the drug 105 through the cannula 106.

As shown in FIG. 11, the open window 110 may be defined in the body 102, as part of the barrel. The body 102 may be opaque about the open window 110. With the body 102 being formed of glass, as is typical with syringe barrel construction, the body 102 may have a decal or coating applied to the exterior thereof which is frosted, or otherwise obscured, to provide opaqueness to the body 102. The open window 110 in reverse has no decal or coating to be clear. This embodiment generally operates in similar manner to the pen injector embodiment described above, with the second marking 114 being located on the plunger 108 to be revealed through the open window 110 post-use.

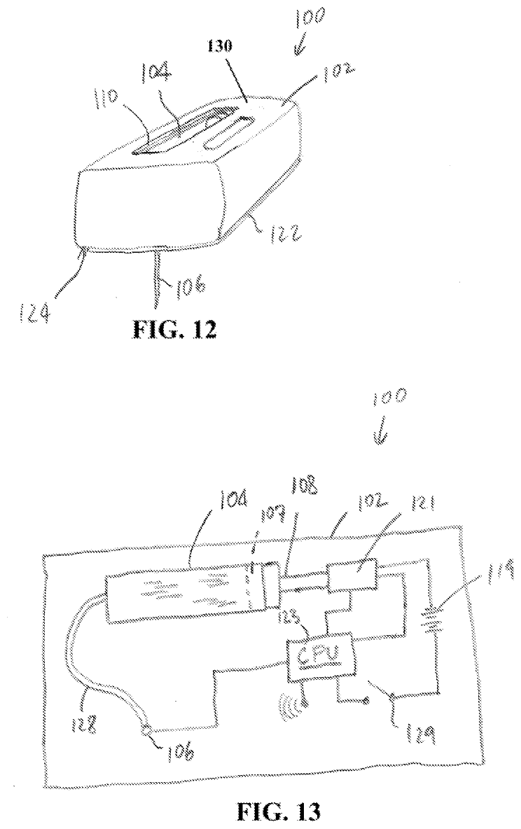

In a further embodiment, the medical injector 100 may be in the form of a body-wearable injector, as shown in FIGS. 12-29. As shown in FIG. 12, the body 102 of the medical injector 100, may be in the form of a housing which may be held against a patient's skin by adhesive, a strap, or other holding arrangement. The body 102 may be box-shaped with a layer of adhesive 122 on a base face 124 thereof. As known in the art, the adhesive 122 is configured to be releasable from the skin post-injection. As shown schematically in FIG. 13, body-wearable injectors are known in the art to have an internal drug reservoir 104 with a plunger 108 for urging drug from the reservoir 104 to cannula 106, via one or more connections (e.g., one or more conduits 128). As known in the art, and discussed above, the reservoir 104 may be sealed at one end by stopper 107. With a typical body-wearable injector, the cannula 106 is hidden within the body 102 prior to use. As shown in FIG. 12, the cannula 106 is caused to extend from the body 102, through the base face 124, with the body 102 secured to the patient. The cannula 106 may be auto-or manually driven to extend from the body 102, as is known in the art. A motor or other drive mechanism 121 may be provided, housed within the body 102, to drive the plunger 108 to dispense the drug through the cannula 106, particularly with the cannula 106 inserted into the patient's skin. Post-injection, the cannula 106 may be retracted (e.g., auto-retracted) into the body 102. A controller 123, such as a computer processing unit or the like, may be provided to control the medical injector 100, including the cannula 106 and/or the motor 121. A supply of electrical energy 119 may be provided, for example in the form of a battery, housed within the body 102, and electrically coupled to provide electrical energy to various components of the medical injector 100, including, but not limited to, the motor 121 and the controller 123. One or more switches 129 may be provided to allow a user to control the medical injector 100, including to initiate activation. Although not shown, the one or more switches 129 are typically externally accessible on the body 102, e.g., in the form of one or more buttons, to allow user interaction. As will be recognized by those skilled in the art, body wearable injectors can include additional or different features beyond those described herein. It is to be understood that the principles of the subject invention would equally apply to differently-configured body-wearable injectors.

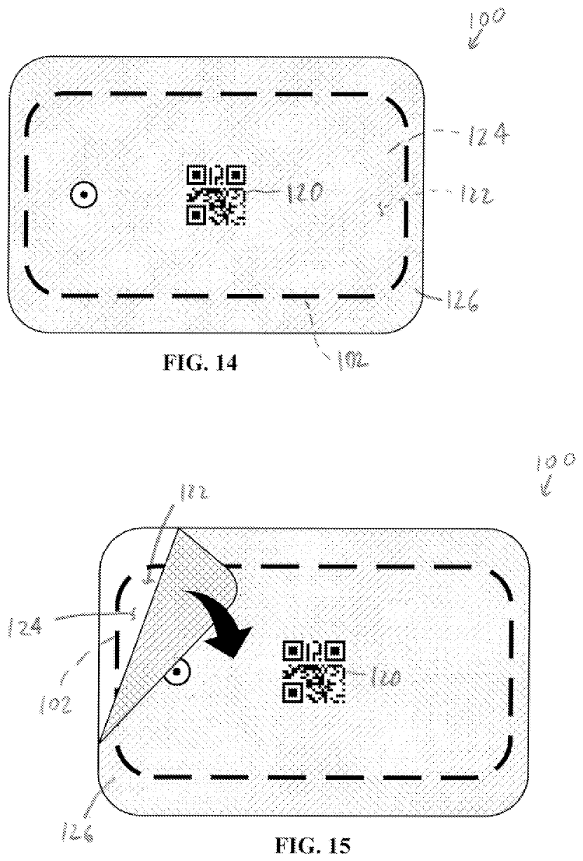

In one variation, as shown in FIGS. 14-16, the first marking 112 may be provided on the body 102. A release liner 126 may be provided to cover the adhesive 122 prior to use. The release liner 126 is peelable to expose the adhesive 122 to allow the body 102 to be adherently applied to the patient's skin at a desired injection site. The first marking 112 may be provided on the adhesive 122 or the adhesive 122 may be discontinuous with the first marking being located in a discontinuity between portions of the adhesive 122.

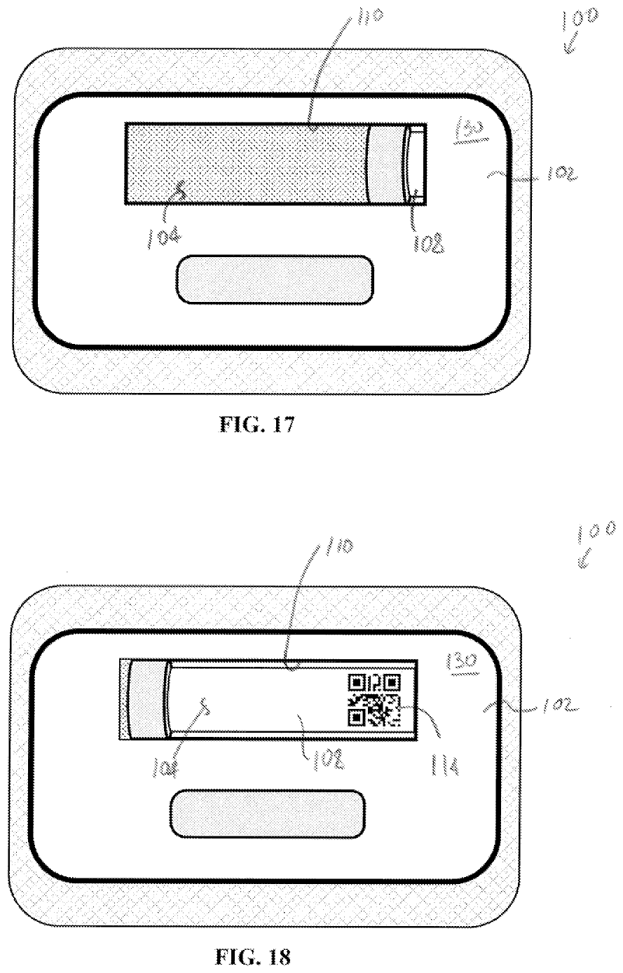

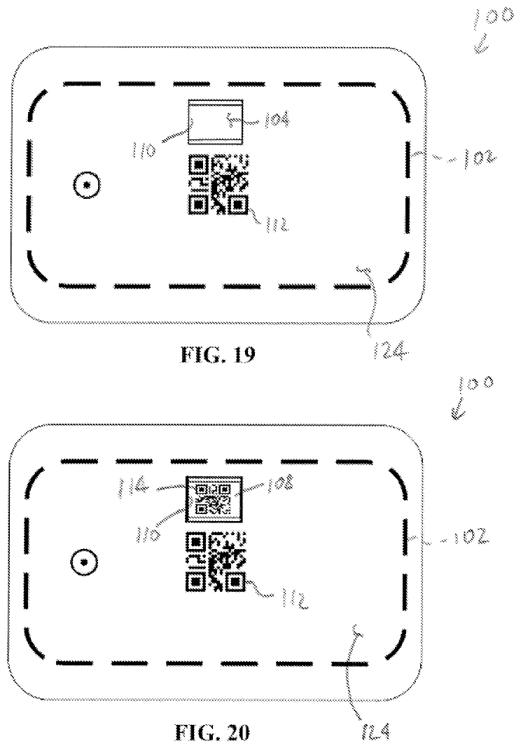

As with other variations of the medical injector 100, open window 110 may be provided on the body 102, and the second marking 114 may be provided on the plunger 108. As shown in FIGS. 17-18, the open window 110 may be located to allow the second marking 114 to come into alignment with the open window 110, so as to be viewable therethrough, upon end-of-injection (i.e., full travel of the plunger 108). The open window 110 may be provided on external face 130 of the body 102, facing in an opposite direction from the base face 124. With this arrangement, the first and second markings 112, 114 would be separately observable (being on opposite faces of the body 102). It is possible to include the first marking 112 on the external face 130 located so as to be in a common view of the camera of the user's device 116. Here, the first marking 112 would be exposed, not being covered by the release liner 126. Alternatively, as shown in FIGS. 19-20, the open window 110 may be provided on the base face 124, e.g., in a discontinuity between portions of the adhesive 122. Here, the second marking 114 may be located initially out of alignment with the open window 110 (FIG. 19) and be located to be aligned with the open window 110 (FIG. 20) at end-of-injection. In addition, the open window 110 may be located on the base face 124 to be in a common view of a camera of the user's device 116 with the first marking 112 (thereby, allowing for the first and second markings 112, 114 to be in a common view of the camera of the user's device 116.) With the open window 110 on the base face 124, the first marking 112 may be hidden prior to use by the release liner 126. As with the other variations of the medical injector 100, the capturing of the first and second markings 112, 114 allows for timestamping (e.g., the beginning and end of an injection), and/or, for providing indication of successful, complete injection.

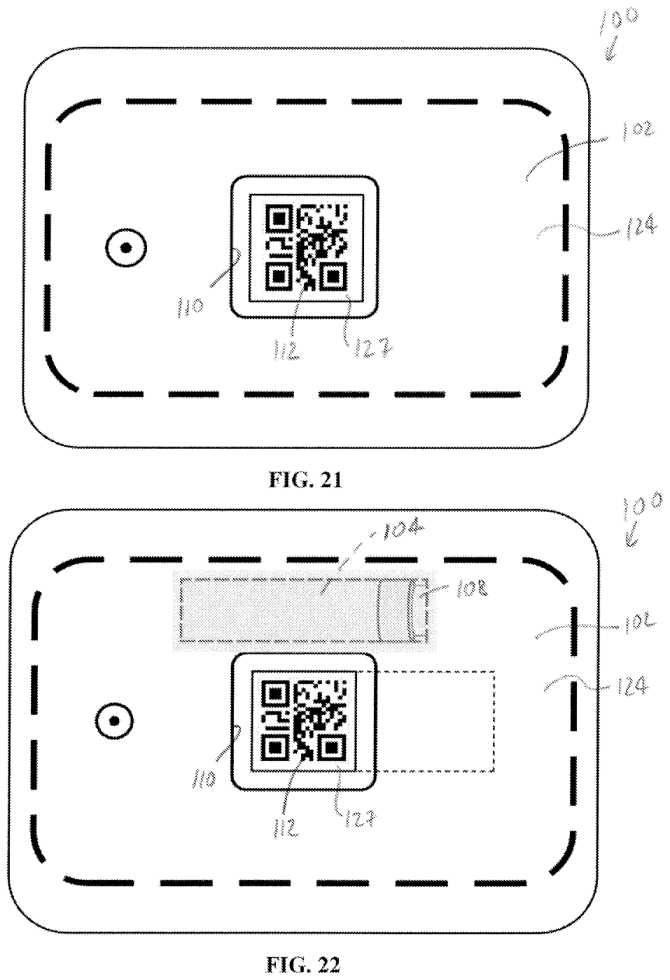

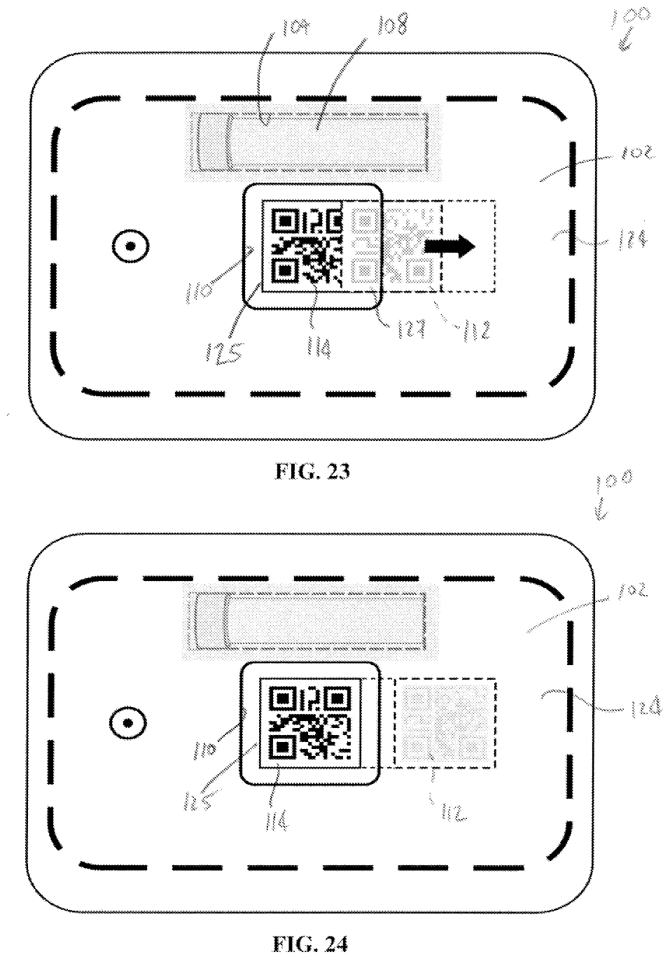

As a further variation, the second marker 114 may be provided on the body 102 separate from the plunger 108, e.g., being provided on a support surface 125 aligned with the open window 110. The support surface 125 may be spaced from the open window 110. A blocking panel 127 is provided with an initial state covering the open window 110 to obscure the second marking 114. The blocking panel 127 is adjustable to a second state, not covering the open window 110 to expose the second marking 114. In one variation, as shown in FIGS. 21-24, the first marking 112 may be provided on the blocking panel 127. The open window 110 is shown to be located on the base face 124, but may be located elsewhere on the body 102. Again, with the open window 110 being located on the base face 124, the first marking 112 may be obscured by the release liner 126 prior to use. As shown in FIG. 21, initially, the first marking 112 is viewable through the open window 110, the initial position coinciding with pre-or start-of-injection (shown schematically in FIG. 22). As shown schematically in FIG. 23, with end-of-injection, the blocking panel 127 is adjusted to the second state. As further shown in FIG. 24, the second marking 114 is revealed and viewable through the open window 110, with the first marking 112 being hidden from view, obscured by a portion of the body 102.

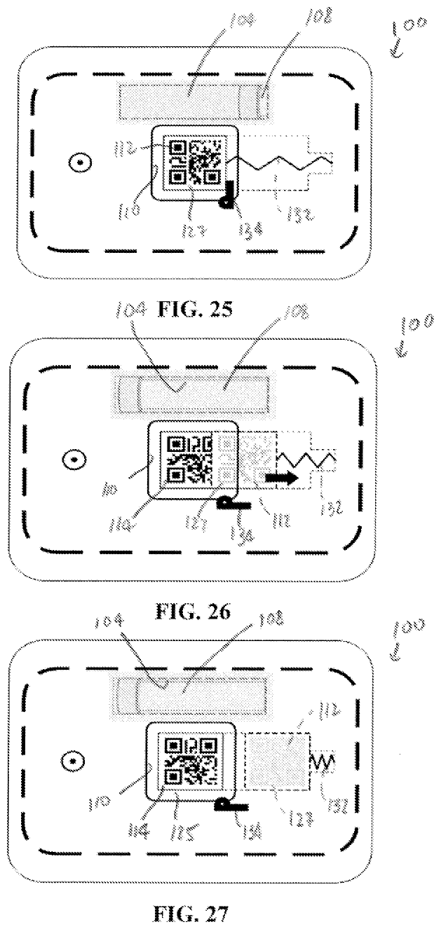

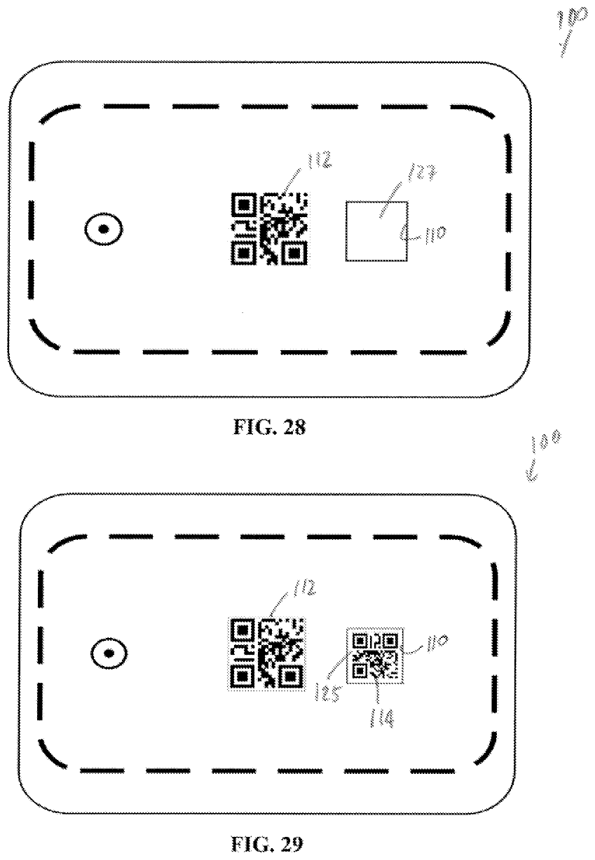

Any arrangement for causing the blocking panel 127 to adjust from the initial state to the second state may be utilized. By way of non-limiting example, as shown in FIGS. 25-27, the blocking panel 127 may be urged, e.g., by spring 132, to the second state, with a retainer 134 holding the blocking panel 127 in the initial state. The retainer 134 is caused to allow release of the blocking panel 127, resulting in adjustment to the second state. The retainer 134 may be in the form of a shiftable or rotatable finger positioned to overlap a portion of the blocking panel 127, as shown in FIG. 25. The blocking panel 127 may be hinged-connected to the body 102 or slidable relative to the open window 110. The retainer 134 acts to restrain rotation or sliding movement of the blocking panel 127 from the initial state, prior to release. As shown in FIGS. 26-27, the retainer 134 may be caused to adjust (shift or rotate) upon completion of the injection (e.g., the plunger 108 has completed its full travel) or upon the cannula 106 being retracted into the body 102. The retainer 134 may be caused to adjust by the controller 123 or a mechanical connection (direct or indirect connection) between the retainer 134 and the plunger 108 or a mechanical connection (direct or indirect connection) between the retainer 134 and a component related to the cannula 106. The noted arrangement, however, does not allow for simultaneous viewing of the first and second markings 112, 114. In an alternative arrangement, as shown in FIGS. 28-29, the open window 110 may be provided separate from the first marking 112. In the same manner as previously described, the blocking panel 127 initially covers the second marking 114 (FIG. 28), and, with release of the blocking panel 127, the second marking 114 is revealed (FIG. 29). As shown in FIG. 29, with this arrangement, the first and second markings 112 and 114 are viewable in a common view of the camera of the user's device 116.

Post-injection, with the cannula 106 retracted in the body 102, and, with removal of the body 102 from the patient's skin, the cannula 106 would be hidden within the body 102. Thus, the cannula 106 would not affect any observations of the first and second markings 112, 114, particularly if viewable on the base face 124.

As shown in FIGS. 14-16, the third marking 120 may be provided on the release liner 126, particularly on an external surface thereof, to be viewable prior to removal of the release liner 126 to expose the adhesive 122. The third marking 120 may be captured prior to use. This allows for an initial reading to confirm a ready state. For example, the third marking 120 may be captured by the camera of the user's device 116 with the third marking 120 being decoded by the application software to obtain start data. This reading may provide a time stamp to indicate a start time. The obtained data may also act as a key required to allow activation of the medical injector 100. The data may be transmitted by the user's device 116 to a receiver on the medical injector 100 (e.g., receiver associated with the controller 123) which then, utilizing the controller 123 allows the medical injector 100 to be activated. The key may act as a fail-safe to prevent premature activation. Subsequently, the first marking 112 and/or the second marking 114 may be utilized to provide data regarding injection conditions (start, completion, and so forth).

As will recognized by those skilled in the art, the medical injector 100, whether in the form of a pen injector, syringe, or body-wearable injector, may utilize the second marking 114, without the first marking 112 and/or the third marking 120. Here, the second marking 114 would be revealed as a result of injection, as discussed above. The second marking 114 alone may be optically read and provide useful information.

### Pill Bottle

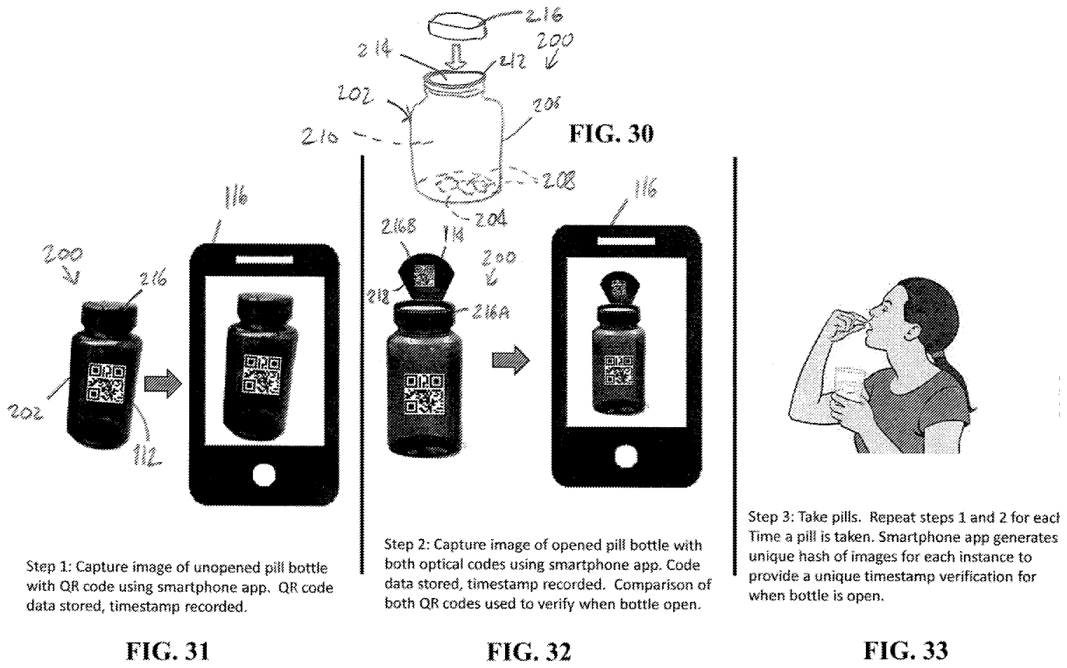

With reference to FIGS. 30-33, the drug delivery device may be in the form of a medical pill bottle 200. The pill bottle 200 includes a standard construction with a reservoir 202 having a base 204 bounded by an upstanding side wall 206. The reservoir 202 defines an interior volume 210 to accommodate a supply of medical pills 208. The side wall 206 terminates at a free end 212 which defines an opening 214 in communication with the interior volume 210. The opening 214 provides access to the interior volume 210 to allow for removal of the medical pills 208.

A cap 216 is mounted to the side wall 214 across the opening 214. The cap 216 is displaceable between an open state, allowing access to the interior volume 210 through the opening 214, and a closed state, fully covering the opening 214. As shown in FIG. 30, the cap 216 may be displaced by removal from the side wall, e.g., with the cap 216 being threadedly mounted to the side wall 206 about the opening 214. The cap 216 may be provided with child-resistant features, as known in the art. Alternatively, as shown in FIGS. 31-32, the cap 216 may be multi-component including a first cap portion 216A, mounted to the side wall 206, and a second cap portion 216B movable relative to the first cap portion 216A to be displaced between the open and closed states. The second cap portion 216B may be hingedly connected to the first cap portion 216A to be rotated between the open and closed states. The first and second cap portions 216A, 216B may be provided with cooperating, releasable engagement members to releasably fix the second cap portion 216B to the first cap portion 216A in the closed state (e.g., cooperating snap engagement features).

The second marking 114 may be provided on a lower surface 218 of the cap 216. In this manner, the second marking 114 is concealed with the cap 216 in the closed state. With the cap 216 in the open state, the second marking 114 may be revealed. With the cap 216 being multi-component, the second marking 114 may be located on the lower surface 218 of the second cap portion 216B. The first marking 112 may be applied to an exterior portion of the reservoir 202. The reservoir 202 may be elongated to extend along a longitudinal axis. It is preferred that the first and second markings 112, 114 be positioned along the longitudinal axis at spaced-apart locations. This allows for good alignment of the first and second markings 112, 114 in a common field of view of a camera of a user's device 116.

In use, an image may be captured of the pill bottle 200 in the closed state, using the user's device 116. The application software may timestamp the captured image. The cap 216 is displaced to the open state (by removal or adjustment of the second cap portion 216B) with one or more of the medical pills 208 removed from the interior portion 210, depending to the required dose. With the cap 216 in the open state, the user's device 216 may be used to capture a post-dose image of the first and second markings 112, 114. The cap 216, if removed, may be placed adjacent to the reservoir 202 to allow for the first and second markings 112, 114 to be in the common field of view of the camera of the user's device 116. The application software may conduct verification of the first and second markings 112, 114, as discussed above. The image may be timestamped. The application software may generate a data packet based on the timestamps and/or verification for transmission to a remote server.

It is understood that with the pill bottle 200, the first and second markings 112, 114 are static, with the same codes being used to verify a plurality of doses. This may allow for falsification of dosing information due to its repeated use. Use of the timestamp, and availability of remaining doses, at a remote location provide some basis to verify legitimacy of collected data through comparison with an expected dosing timetable. In addition, or alternatively, a hash of each captured image may be also computed, thus generating a unique hash for each opening of the pill bottle 200 (each opening representing a separate dosing). The generated hashes could be used to verify that the pill bottle 200 has been opened that number of times (at least, openings for which images were not captured are not detectable but this could be addressed by reconciliation errors in a count of the medical pills 208, assuming the medical pills 208 were consumed on the uncaptured openings). Such reconciliation errors could be used to detect non-compliance and trigger interventions.

### Blister Pack

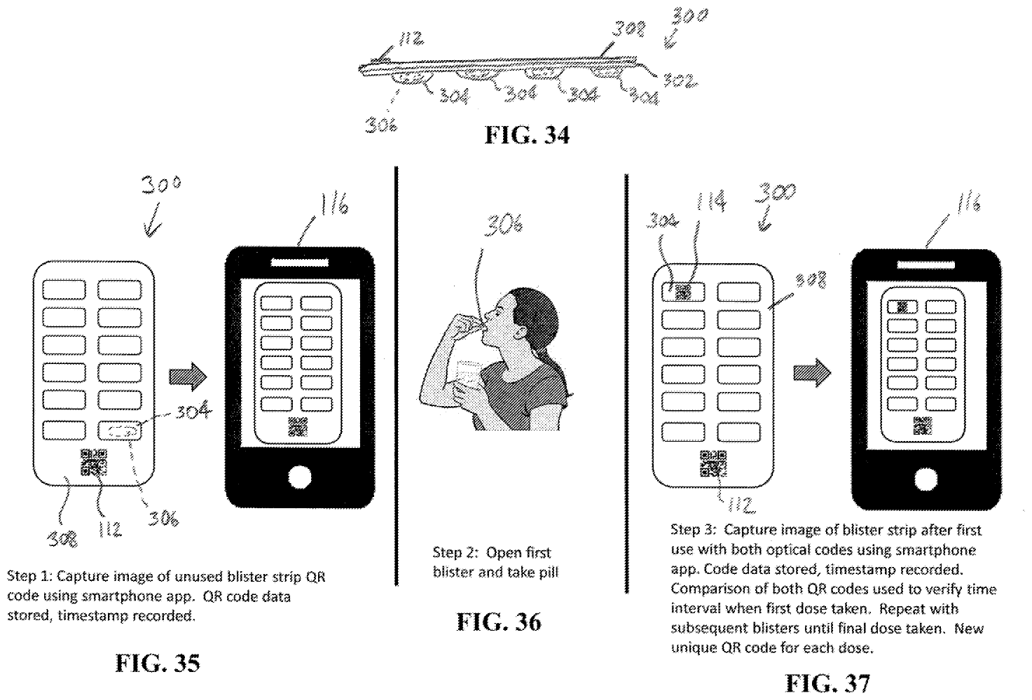

With reference to FIGS. 34-37, the drug delivery device may be in the form of a medical pill blister pack 300. The blister pack 300 includes standard construction with a plastic layer 302 formed to define an array of blisters 304. Each of the blisters 304 is formed to accommodate at least one medical pill 306. The plastic layer 302 may be formed using any technique including injection molding, thermoforming, and the like. A foil layer 308 is secured to the plastic layer 302 to overlie the blisters 304. The foil layer 308 may be of any material suitable to seal the blisters 304, including, but not limited to, coated paper and aluminum. The foil layer 308 may be fixed to the plastic layer 302 using any known technique, including, but not limited to, adherence, heat welding, and so forth. As known in the art, the foil layer 308 may be thinned or scored about the blisters 304, and/or provided with child-resistant features to limit accessing the blisters 304.

As shown in FIG. 35, the foil layer 308 includes areas out of alignment with the blisters 304. The first marking 112 is located on the exterior of the foil layer 308 out of alignment with the blisters 304. In this manner, the first marking 112 remains intact as the medical pills 306 are successively removed from the blisters 304.

As shown in FIG. 37, the second marking 114 is provided in each of the blisters 304, particularly, at the bottom thereof. With this arrangement, the second marking 114 is revealed with the accessing of the respective blister 304 and the removal of the medical pill 306 therefrom. With the pill bottle 200, the second marking 114 is revealed with the opening thereof. In contrast, with the blister pack 300, both the blister 304 needs to be accessed and the medical pill 306 must be removed to reveal the second marking 114. This provides for greater integrity in ensuring drug administration.

In use, the user's device 116 is used to capture an image of the first marking 112 prior to accessing one of the blisters 304 for dosing. This image may be timestamped. With the medical pill 306 removed, it is taken by a patient, as shown in FIG. 36. Subsequently, post-dose image is captured by the user's device 116, with the first and second markings 112, 114 being in common field of view of the camera of the user's device 116, as shown in FIG. 37. This image may be likewise timestamped. The application software may conduct verification of the first and second markings 112, 114, as discussed above. The application software may generate a data packet based on the timestamps and/or verification for transmission to a remote server.

It is understood that with the pill bottle 200, the first and second markings 112, 114 are static, with the same codes being used to verify a plurality of doses. This may allow for falsification of dosing information due to its repeated use. Use of the timestamp, and availability of remaining doses, at a remote location provide some basis to verify legitimacy of collected data through comparison with an expected dosing timetable. In addition, the second marking 114 may be provided in each of the blisters 304. Thus, with multiple blisters 304 having been accessed, multiple second markings 114 may be in the common view of the camera of the user's device 116. The multiple second markings 114 may be hashed as discussed above to create a hash chain, providing a combination of the same code. Alternatively, the second markings 114 may be each altered so that each of the blisters 304 has a unique second marking associated therewith. The second markings 114 may be altered to have a data bit or string in addition to a common unique number, with the common unique number being useable with the algorithmic operations for verification against the first marking 112. The data bit or string may be used a serialization character to count individual doses. It is noted that the software application may prompt the user to cover, remove or otherwise obscure older revealed second markings 114 so that the latest second marking 114 is only shown in the common field of view of the camera of the user's device 116.

### System

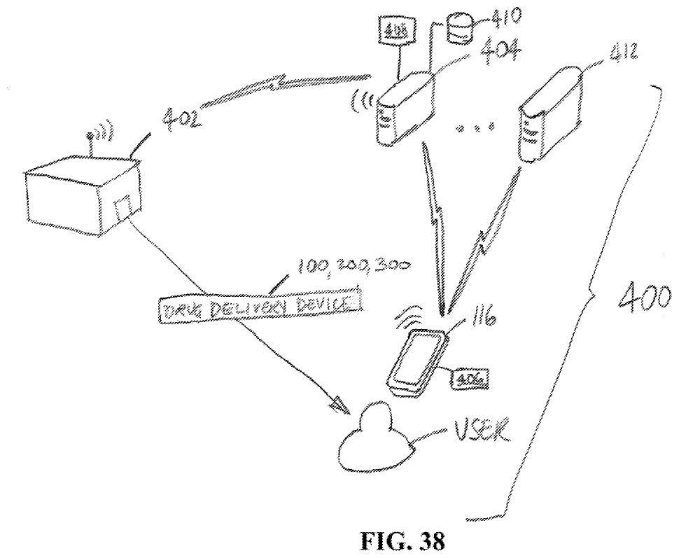

With reference to FIG. 38, a drug delivery device formed in accordance with the subject invention may be used as part of a system 400. The system 400 may include interactions with a manufacturing facility 402 where the drug delivery device is manufactured and/or, in whole or in part. As discussed above, the first marking 112 and/or the second marking 114 (and any other concealed, to-be-revealed markings) may be applied to the drug delivery device at the facility 402. Components may be provided to the facility 402 pre-marked (to be optically read during assembling) and/or marked at the facility 402. A control server 404 may control the generation of the creation of the first and second markings 112, 114 based on selected algorithmic operation(s). The algorithmic operation(s) may be static (unchanging for all components), with the application software 406 being programmed with the selected algorithmic operation(s). The control server 404 has a non-transitory memory 408 and/or database 410 associated therewith, on which associations between the first marking 112, the second marking 114, the selected algorithmic operation(s), and any other used, markings, may be stored. The associations may be saved against details of the drug delivery devices, such as product numbers, batch numbers, date of assembly, drug details (type(s), concentration(s)), and date of drug expiration. The associations may also include unique numbers generated for any of the markings

The control server 404, although referenced in the single, may be a series of distributed computer processing units, located at the same location or remotely. The computer processing units may be connected by local area network (LAN), wide area network (WAN) and/or over the Internet. Likewise, the memory 408 and the database 410, although referenced in the single, may be distributed over a series of linked memories and/or databases.

The application software 406 is provided to be downloaded from a portal, controlled by a user of the system 400, and/or from an application software marketplace, such as Google Play or Apple App Store. The application software 406 may be configured to reside on the user's device 116, with the ability to make calls to a remote server to retrieve information (e.g., API calls). The application software 406 may be also configured to transmit collected data (e.g., wirelessly) to the control server 404 and/or a secondary server 412 for collected data to be reviewed and analyzed. The control server 404 and/or the secondary server 412 may be operated by, or accessed by (subject to privacy laws and regulations), one or more of: the manufacturer of the drug delivery device, the drug manufacturer, a healthcare provider, a medical insurance provider, and/or, a compliance organization.

1. A medical injector comprising:
a body;
a drug reservoir contained within, or defined by, the body, the drug reservoir configured to accommodate a supply of drug;
a cannula;
a movable plunger for urging drug from the drug reservoir through the cannula;
a first optically readable code on the body; and,
a second optically readable code on the plunger,
wherein, an open window is formed in the body,
wherein, the plunger is initially in a first state with the second optically readable code out of alignment with the open window, wherein, with the plunger in the first state, the second optically readable code is concealed within the body, and,
wherein, the plunger moves to a second state from the first state to urge drug from the drug reservoir through the cannula, the second optically readable code moving with the plunger with the plunger moving from the first state to the second state, and, wherein, with the plunger in the second state, the second optically readable code is in alignment with the open window so as to be viewable therethrough.

2. The medical injector of claim 1, wherein the body is elongated extending along a longitudinal axis, the first optically readable code and the second optically readable code being positioned along the longitudinal axis.

3. The medical injector of claim 1, further comprising a needle shield formed to cover the cannula, a third optically readable code being located on the needle shield, wherein, the third optically readable code is located on the needle shield so as to be covered by the body whilst the plunger moves to the second state from the first state, and, wherein, the needle shield is configured to extend from the body in a fully extended state to cover the cannula, the third optically readable code being exposed with the needle shield in the fully extended state.

4. The medical injector of claim 1, wherein a clear or translucent lens is located within the open window.

5. The medical injector of claim 1, wherein, with the plunger in the second state, the first optically readable code and the second optically readable code are positioned to be both viewable in a common field of view of a camera of a smartphone.

6. The medical injector of claim 1, wherein the body about the first optically readable code is in a contrasting color from the plunger about the second optically readable code.

7. The medical injector of claim 1, wherein first data is encoded within the first optically readable code to represent at least one of: the drug; strength of the drug; manufacturing batch number; time of manufacturing the drug; date of manufacturing the drug; time of assembling the medical injector; date of assembling the medical injector; expiration date of the drug; and, a unique number.

8. The medical injector of claim 1, wherein a first number is encoded within the first optically readable code and a second number is encoded within the second optically readable code, and, wherein, the second number is algorithmically related to the first number.

9. The medical injector of claim 1, wherein the drug reservoir is in the form of an ampoule, the ampoule have a clear or translucent side wall.

10.-27. (canceled)

28. A medical injector comprising:
a body;
a drug reservoir contained within, or defined by, the body, the drug reservoir configured to accommodate a supply of drug;
a cannula;
a movable plunger for urging drug from the drug reservoir through the cannula; and, an optically readable code on the plunger,
wherein, an open window is formed in the body,
wherein, the plunger is initially in a first state with the optically readable code out of alignment with the open window, wherein, with the plunger in the first state, the optically readable code is concealed within the body, and,
wherein, the plunger moves to a second state from the first state to urge drug from the drug reservoir through the cannula, the optically readable code moving with the plunger with the plunger moving from the first state to the second state, and, wherein, with the plunger in the second state, the optically readable code is in alignment with the open window so as to be viewable therethrough.

29. The medical injector of claim 28, wherein the medical injector is selected from: a pen injector, a syringe, and a body-wearable injector.

30. The medical injector of claim 29, wherein, with the medical injector being a body-wearable injector, the body is box-shaped with a base face, with a layer of adhesive associated therewith, and an external face facing in an opposite direction from the base face.

31. The medical injector of claim 30, wherein the open window is formed in the base face.

32. The medical injector of claim 31, wherein a second optically readable code is located on the layer of adhesive or in a discontinuity formed in the layer of adhesive.

33. The medical injector of claim 30, wherein the open window is formed in the external face.

34. A system to monitor the administration of drug to a patient, the system comprising:
a medical injector as set forth in claim 1; and,
a camera-equipped, Internet enabled device having stored thereon application software, wherein, the camera of the device is configured to read the first and second optically readable codes, wherein, the application software is configured to: i. conduct one or more algorithmic operations to determine, based on one of the first optically readable code and the second optically readable code, an intermediate code; and, ii. compare the intermediate code against the other of the first optically readable code and the second optically readable code for verification.

35. The system of claim 34, wherein first data is encoded within the first optically readable code to represent at least one of: the drug; strength of the drug; manufacturing batch number; time of manufacturing the drug; date of manufacturing the drug; time of assembling the medical injector; date of assembling the medical injector; expiration date of the drug; and, a unique number.

36. The system of claim 34, wherein a first number is encoded within the first optically readable code and a second number is encoded within the second optically readable code, the second number being algorithmically related to the first number.

37. The system of claim 34, wherein the second number is algorithmically related to the first number by one or more mathematical operations.

38. The system of claim 37, wherein the one or more mathematical operations may include at least one selected from the group consisting of: addition of a predetermined value; subtraction of a predetermined value; multiplication by a predetermined value; and, division by a predetermined value.

39. The system of claim 34, wherein, at least one of the first optically readable code and the second optically readable code includes an encoded data bit or data string representative of the one or more algorithmic operations, and, wherein, the application software being configured to determine the one or more algorithmic operations based on the data bit or data string.

40. The system of claim 39, wherein the application software is configured to call a remote database to look up the one or more algorithmic operations based on the data bit or data string.

41. The system of claim 39, wherein the application software is associated with a look-up table cross-referencing the data bit or data string against the one or more algorithmic operations.

42.-47. (canceled)
